(19) United States
(12) Patent Application Publication
(10) Pub. No.: US 2026/0367385 A1
(43) Pub. Date: Nov. 19, 2026

(54) ILLUMINATION LENS AND ENDOSCOPE

(71) Applicant: FUJIFILM Corporation, Tokyo (JP)

(72) Inventor: Keisuke HARADA, Saitama (JP)

(73) Assignee: FUJIFILM Corporation, Tokyo (JP)

(21) Appl. No.: 19/651,767

(22) Filed: Apr. 19, 2026

(30) Foreign Application Priority Data
Apr. 24, 2025 (JP) 2025-072680

(51) Int. Cl. G02B 23/24; A61B 1/00; A61B 1/07; G02B 3/04; G02B 5/02

(52) U.S. Cl. CPC G02B 23/2423; A61B 1/00096; A61B 1/07; G02B 3/04; G02B 5/0215; G02B 5/0294; G02B 23/2461

(57) ABSTRACT

An illumination lens that is disposed at a distal end of a light guide of an endoscope, the illumination lens consisting of one negative lens, wherein: a first lens surface of the illumination lens on a light guide side includes a concave surface that is provided at a center portion thereof and a light diffusion surface that is provided outside the concave surface in a radial direction, and in a case where a refractive index of the illumination lens at a d line is denoted by Nd, a diameter of the light guide is denoted by G, and a distance on an optical axis from an emission end surface of the light guide to a second lens surface of the illumination lens on an irradiated object side is denoted by D2, Conditional Expression (1) is satisfied: 3.55<Nd×G/D2<10 (1).

### CROSS-REFERENCE TO RELATED APPLICATIONS

This application claims priority from Japanese Application No. 2025-072680, filed on Apr. 24, 2025, the entire disclosure of which is incorporated herein by reference.

### BACKGROUND

### Technical Field

A technology of the present disclosure relates to an illumination lens and an endoscope.

### Related Art

In the related art, for example, illumination optical systems disclosed in JP5307957B, JP1993-53063A (JP-H05-53063A) and JP2003-131144A are known as an illumination optical system that is disposed at a distal end part of an insertion part of an endoscope and is used to illuminate an object to be examined.

In recent years, there has been a demand for an illumination lens that is configured to be small and has wide light distribution and good transmission efficiency.

### SUMMARY

The present disclosure provides an illumination lens that is configured to be small and has wide light distribution and good transmission efficiency, and an endoscope including the illumination lens.

According to an aspect of the disclosed technology, there is provided an illumination lens that is disposed at a distal end of a light guide of an endoscope, the illumination lens consisting of one negative lens, in which a first lens surface of the illumination lens on a light guide side includes a concave surface that is provided at a center portion thereof and a light diffusion surface that is provided outside the concave surface in a radial direction, and

Conditional Expression (1) is satisfied, which is represented by

3.55<Nd×G/D2<10 (1).

Here, a refractive index of the illumination lens at a d line is denoted by Nd. A diameter of the light guide is denoted by G. A distance on an optical axis from an emission end surface of the light guide to a second lens surface of the illumination lens on an irradiated object side is denoted by D2.

It is preferable that the illumination lens according to the above aspect satisfies at least one of Conditional Expressions (2) to (10). Here, a paraxial curvature radius of the first lens surface is denoted by R1. A distance on the optical axis from the emission end surface of the light guide to the first lens surface is denoted by D1. An Abbe number of the illumination lens based on the d line is denoted by vd. An outermost diameter of the illumination lens is denoted by Y. A paraxial curvature radius of the second lens surface is denoted by R2. A height from the optical axis, denoted by H1, is defined as H1=0.85×G/2. An angle with respect to the optical axis at which a ray emitted parallel to the optical axis from the emission end surface of the light guide at the height H1 exits the second lens surface is denoted by φ2. A unit of φ2 is radian. A height of the ray from the optical axis at the second lens surface, the ray being emitted parallel to the optical axis from the emission end surface of the light guide at the height H1, is denoted by H2. A center thickness of the illumination lens is denoted by T. A distance in a direction of the optical axis from an intersection of the ray emitted parallel to the optical axis from the emission end surface of the light guide at the height H1 with the first lens surface to an intersection of the ray with the second lens surface is denoted by E.

$$0.82 < \frac{2 \sqrt{{\vert R 1 \vert}^{2} - {( \vert R 1 \vert - D 1 )}^{2}}}{G} < 0.95 \qquad ( 2 )$$

$$2.3 < Nd + 0.0 \; 115 \times ν d < 2.55 \qquad ( 3 )$$

$$3.2 < Nd - 0.0 \; 01 \times ν {d}^{2} + 0.075 \times ν d < 3.6 \qquad ( 4 )$$

$$5 < Nd \times Y / D 2 < 20 \qquad ( 5 )$$

$$1 \leq ( R 1 + R 2 ) / ( R 2 - R 1 ) < 1.26 \qquad ( 6 )$$

$$0.25 < φ 2 \times H 1 / \vert R 1 \vert < 0.8 \qquad ( 7 )$$

$$1.15 < H 2 / H 1 < 1.8 \qquad ( 8 )$$

$$0.5 < 2 \times H 2 / Y < 0.99 \qquad ( 9 )$$

$$1.3 < E / T < 1.9 \qquad ( 10 )$$

Another aspect of the disclosed technology is an endoscope comprising the illumination lens according to the above aspect.

“Consisting of” and “consist of” in this specification may intend to include: a lens substantially not having optical power; optical elements other than the lens, such as a stop, a filter, and a cover glass; a lens flange; a lens barrel; and the like in addition to mentioned components.

In the present specification, the terms “lens having a negative optical power” and “negative lens” are synonymous. The term “single lens” means one non-cemented lens. The number of lenses described above is the number of lenses as constituent elements. For example, it is assumed that the number of lenses in a cemented lens in which a plurality of single lenses having different materials are cemented is represented by the number of single lenses constituting the cemented lens. However, a compound aspherical lens (that is, a lens of which a lens (for example, a spherical lens) and an aspherical film formed on the lens are integrally formed and which functions as one aspherical lens as a whole) is not regarded as a cemented lens, but is regarded as one lens.

A sign of the optical power of a lens that includes an aspherical surface, the curvature radius of a surface of the lens, and the shape of a surface of the lens are considered in a paraxial region unless otherwise specified. A sign of a curvature radius is positive for a surface with a convex shape toward a light guide side and a sign of a curvature radius is negative for a surface with a convex shape toward an irradiated object side.

Unless otherwise specified, the expression “distance on the optical axis” used in the conditional expressions means a geometrical distance. A value used in Conditional Expression is a value with respect to the d line (a wavelength of 587.56 nanometers (nm)).

In the description of the present specification, the term “parallel” refers to not only perfect parallelism but also approximate parallelism including an error generally allowed in the technical field to which the present disclosed technology belongs. The term “plane” refers to not only a perfect plane but also an approximate plane including an error generally allowed in the technical field to which the present disclosed technology belongs.

According to the present disclosure, it is possible to provide an illumination lens that is configured to be small, and has wide light distribution and good transmission efficiency, and an endoscope including the illumination lens.

### BRIEF DESCRIPTION OF THE DRAWINGS

FIG. 1 is a cross-sectional view showing a configuration of an illumination lens according to an embodiment, which corresponds to an illumination lens of Example 1, and an optical path of a ray incident on a concave surface.

FIG. 2 is a diagram showing a light diffusion surface.

FIG. 3 is a diagram showing a comparative example in which a light diffusion surface is not provided.

FIG. 4 is a diagram for describing symbols of each conditional expression.

FIG. 5 is a partially enlarged view for describing the symbols of the conditional expressions.

FIG. 6 is a cross-sectional view showing a configuration of an illumination lens of Example 1 and an optical path of a ray incident on a light diffusion surface.

FIG. 7 is a cross-sectional view showing the configuration of the illumination lens of Example 1 and an optical path of a ray emitted at a height H1.

FIG. 8 is a cross-sectional view showing a configuration of an illumination lens of Example 2 and an optical path of a ray incident on a concave surface.

FIG. 9 is a cross-sectional view showing the configuration of the illumination lens of Example 2 and an optical path of a ray incident on a light diffusion surface.

FIG. 10 is a cross-sectional view showing the configuration of the illumination lens of Example 2 and an optical path of a ray emitted at a height H1.

FIG. 11 is a cross-sectional view showing a configuration of an illumination lens of Example 3 and an optical path of a ray incident on a concave surface.

FIG. 12 is a cross-sectional view showing the configuration of the illumination lens of Example 3 and an optical path of a ray incident on a light diffusion surface.

FIG. 13 is a cross-sectional view showing the configuration of the illumination lens of Example 3 and an optical path of a ray emitted at a height H1.

FIG. 14 is a cross-sectional view showing a configuration of an illumination lens of Example 4 and an optical path of a ray incident on a concave surface.

FIG. 15 is a cross-sectional view showing the configuration of the illumination lens of Example 4 and an optical path of a ray incident on a light diffusion surface.

FIG. 16 is a cross-sectional view showing the configuration of the illumination lens of Example 4 and an optical path of a ray emitted at a height H1.

FIG. 17 is a cross-sectional view showing a configuration of an illumination lens of Example 5 and an optical path of a ray incident on a concave surface.

FIG. 18 is a cross-sectional view showing the configuration of the illumination lens of Example 5 and an optical path of a ray incident on a light diffusion surface.

FIG. 19 is a cross-sectional view showing the configuration of the illumination lens of Example 5 and an optical path of a ray emitted at a height H1.

FIG. 20 is a cross-sectional view showing a configuration of an illumination lens of Example 6 and an optical path of a ray incident on a concave surface.

FIG. 21 is a cross-sectional view showing the configuration of the illumination lens of Example 6 and an optical path of a ray incident on a light diffusion surface.

FIG. 22 is a cross-sectional view showing the configuration of the illumination lens of Example 6 and an optical path of a ray emitted at a height H1.

FIG. 23 is a diagram showing a schematic configuration of an endoscope according to an embodiment.

### DETAILED DESCRIPTION

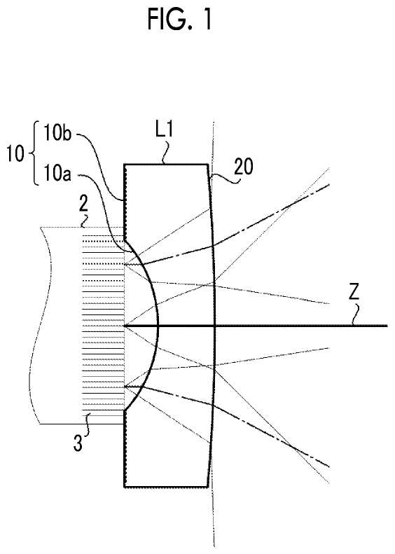

Hereinafter, embodiments of the present disclosure will be described in detail with reference to the accompanying drawings. FIG. 1 is a cross-sectional view showing a configuration and an optical path of an illumination lens L1 according to an embodiment of the present disclosure. The optical path shown in FIG. 1 is an optical path of rays in a case where a plurality of rays emitted from a plurality of points on an emission end surface of a light guide 2 are incident on a concave surface 10a of the illumination lens L1. The illumination lens L1 shown in FIG. 1 corresponds to Example 1 described below.

The illumination lens L1 is disposed at a distal end of the light guide 2 of the endoscope. Specifically, an edge surface of the illumination lens L1 is disposed to be in close contact with or close to a distal end of the light guide 2 on an emission side. The light guide 2 consists of a bundle fiber in which a plurality of optical fibers 3 are bundled, and emits light emitted from a light source (not shown) to the illumination lens L1. That is, the light emitted from the light source is incident on the illumination lens L1 through the light guide 2 and is emitted from the illumination lens L1 to be illumination light. In a case where the illumination lens L1 is disposed at a distal end part of an insertion part of the endoscope, the illumination light illuminates an irradiated object (not shown) that is an observation target. FIG. 1 is a cross-sectional view which includes an optical axis Z and in which a left side is a light source side and a right side is an irradiated object side.

The illumination lens L1 of the present disclosure consists of one negative lens. Since the illumination lens L1 consists of one lens, there is an advantage in suppressing a decrease in transmission efficiency of light.

A first lens surface 10 of the illumination lens L1 on a light guide 2 side includes a concave surface 10a at a center portion and a light diffusion surface 10b on a radial outside of the concave surface 10a. Since the center portion of the first lens surface 10 is formed of the concave surface 10a, it is easy to increase a light distribution angle while achieving a reduction in size. Since the light diffusion surface 10b is provided outside the concave surface 10a of the first lens surface 10 in the radial direction, it is easy to improve the transmission efficiency of light and to maintain a wide light distribution angle (details will be described later).

Here, the light diffusion surface 10b will be described in detail. In a case where a diameter of the concave surface of the first lens surface 10 is equal to or larger than a diameter of an emission end surface of the light guide 2, light emitted from a region on the radial outside of the emission end surface of the light guide 2 is diverged in a case of passing through the concave surface, which causes light leakage. Further, since a change in the depth of the concave surface is steep toward the outer periphery, an area of the concave surface through which the light passes is increased. Therefore, in the concave surface, an effect of light leakage and absorption of light by a chamfered portion on the transmission efficiency is larger at the outer peripheral portion than at the center portion.

In the illumination lens L1 according to the present embodiment, the light diffusion surface 10b is formed on a plane extending from the concave surface 10a toward the radial outside in the first lens surface 10 on the light guide 2 side. That is, since the light diffusion surface 10b is formed outside the outer peripheral portion of the concave surface 10a that is likely to cause the transmission efficiency of light to be reduced, it is possible to suppress a reduction in the transmission efficiency of light.

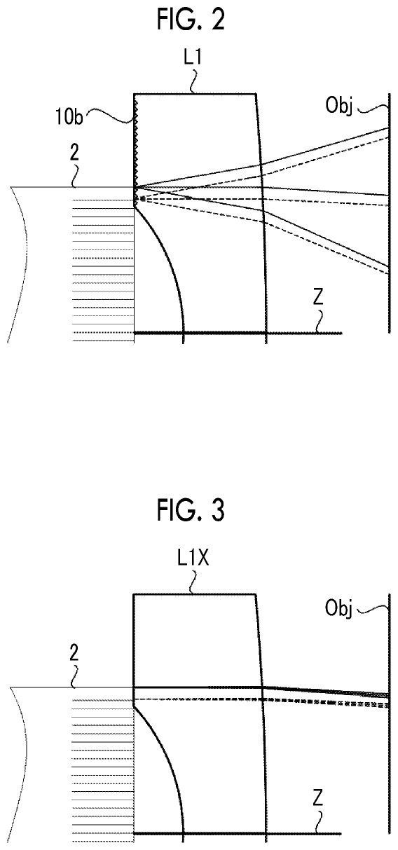

FIG. 2 is a diagram showing a state of a ray emitted from the emission end surface of the light guide 2, incident on the light diffusion surface 10b of the illumination lens L1 of FIG. 1, and emitted from the illumination lens L1. FIG. 3 is a diagram showing a state of a ray in a case where the illumination lens L1 of FIG. 1 is replaced with a lens L1X not having the light diffusion surface 10b, which is a comparative example of FIG. 2. In FIGS. 2 and 3, rays incident on the illumination lens L1 or the lens L1X from two points on the emission end surface of the light guide 2 are shown by a straight line and a broken line, respectively.

As shown in FIG. 2, rays having passed through the light diffusion surface 10b are diffused and are projected onto an object surface Obj in a state where the spread of light is large. That is, as in the illumination lens L1 according to the present embodiment, by diffusing the ray by the light diffusion surface 10b, it is easy to keep the light distribution angle wide.

Since the clad not emitting light, gaps between the fibers, and the core emitting light are arranged in the same plane on the emission end surface of the light guide 2 consisting of a bundle fiber, the emission end surface includes dark portions and bright portions. In a case where an image of the emission end surface of the light guide 2 is formed on the object surface Obj, a bright-dark pattern is projected onto the object surface Obj, and significant light distribution unevenness occurs. In a case where the projection image of the pattern of light and shade is clear, there is a concern that the observation of the irradiated object may be hindered. As in the illumination lens L1 according to the present embodiment, by diffusing the ray by the light diffusion surface 10b, it is possible to suppress the occurrence of light distribution unevenness on the object surface Obj.

On the other hand, it can be seen that the degree of diffusion of rays in the lens L1X not including the light diffusion surface 10b as shown in FIG. 3 is lower than that in the case of FIG. 2 and the light is projected onto the object surface Obj in a state where the spread of the light is small. That is, in the lens L1X that does not have the light diffusion surface 10b, it is difficult to keep the light distribution angle wide as compared with the illumination lens L1 according to the present embodiment, and the possibility that light distribution unevenness occurs on the object surface Obj is increased.

The light diffusion surface 10b may be a roughened surface having fine unevenness, and may be, for example, a surface subjected to a sandblasting treatment by polishing. In addition, the light diffusion surface 10b may be a surface on which a layer containing a substance having a light diffusion function, such as glass beads, is provided. The surface roughness of the light diffusion surface 10b having fine unevenness can be typically set to a few m or less in terms of arithmetic average roughness Ra.

Next, a preferable configuration and a possible configuration related to conditional expressions will be described. In the description of the following conditional expressions, in order to avoid lengthy description, the same symbols will be used for terms having the same definition and repeated description of the symbols will be partially omitted.

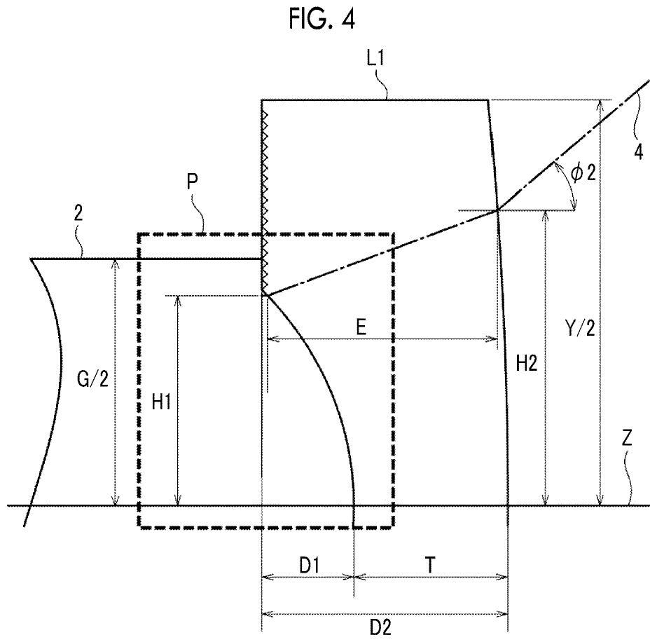

The illumination lens L1 preferably satisfies Conditional Expression (1). Here, a refractive index of the illumination lens L1 at a d line is represented by Nd. A diameter of the light guide 2 is represented by G. A distance on the optical axis Z from an emission end surface of the light guide 2 to a second lens surface 20 of the illumination lens L1 on the irradiated object side is represented by D2. FIG. 4 shows a partially enlarged view of the light guide 2 and the illumination lens L1 of FIG. 1, and shows G/2, which is half of the diameter G (that is, a radius of the light guide 2), and the distance D2 as an example. Ensuring that a corresponding value of Conditional Expression (1) is not less than or equal to its lower limit value prevents an excessively small refractive index and thus, achieves an advantage in achieving a wide-angle light distribution. Ensuring that the corresponding value of Conditional Expression (1) is not greater than or equal to its upper limit value prevents an excessively small thickness of the illumination lens L1 and thus, makes breakage less likely to occur. In addition, since the diameter of the light guide 2 is not excessively large, the blocking of rays on an outer peripheral surface is suppressed and thus, an advantage in improving transmission efficiency is achieved.

3.55<Nd×G/D2<10 (1)

In order to obtain more favorable characteristics, the lower limit value of Conditional Expression (1) is more preferably 3.7, further preferably 3.85, and further preferably 4. In order to obtain more favorable characteristics, the upper limit value of Conditional Expression (1) is more preferably 8.5, further preferably 7, and further preferably 5.5. For example, it is more preferable that the illumination lens L1 satisfies at least one of Conditional Expression (1-1), (1-2), or (1-3).

3.7<Nd×G/D2<8.5 (1-1)

3.85<Nd×G/D2<7 (1-2)

4<Nd×G/D2<5.5 (1-3)

It is preferable that the illumination lens L1 satisfies Conditional Expression (2). Here, it is assumed that a paraxial curvature radius of the first lens surface 10 is R1. It is assumed that a distance on the optical axis Z from the emission end surface of the light guide 2 to the first lens surface 10 is D1. For example, FIG. 4 shows the distance D1. Since a corresponding value of Conditional Expression (2) is made to be larger than a lower limit, the concave surface 10a is not too small. Therefore, light distribution is not excessively narrowed, so that there is an advantage in achieving a wide-angle light distribution. Since the corresponding value of Conditional Expression (2) is made to be smaller than an upper limit, the concave surface 10a is not too large. Therefore, transmission efficiency is not excessively reduced, so that there is an advantage in improving transmission efficiency.

$$0.82 < \frac{2 \sqrt{{\vert R 1 \vert}^{2} - {( \vert R 1 \vert - D 1 )}^{2}}}{G} < 0 \text{.95} \qquad ( 2 )$$

In order to obtain more good characteristics, the lower limit value of Conditional Expression (2) is more preferably 0.83, still more preferably 0.84, and even more preferably 0.85. In order to obtain better characteristics, the upper limit of Conditional Expression (2) is more preferably 0.94, still more preferably 0.93, and even more preferably 0.92. For example, it is more preferable that the illumination lens L1 satisfies at least one of Conditional Expression (2-1), (2-2), or (2-3).

$$0.83 < \frac{2 \sqrt{{\vert R 1 \vert}^{2} - {( \vert R 1 \vert - D 1 )}^{2}}}{G} < 0 \text{.94} \qquad ( 2 - 1 )$$

$$0.84 < \frac{2 \sqrt{{\vert R 1 \vert}^{2} - {( \vert R 1 \vert - D 1 )}^{2}}}{G} < 0.93 \qquad ( 2 - 2 )$$

$$0.85 < \frac{2 \sqrt{{\vert R 1 \vert}^{2} - {( \vert R 1 \vert - D 1 )}^{2}}}{G} < 0.92 \qquad ( 2 - 3 )$$

It is preferable that the illumination lens L1 satisfies Conditional Expression (3). Here, an Abbe number of the illumination lens L1 based on a d line is denoted by vd. By not allowing the corresponding value of Conditional Expression (3) to be equal to or less than the lower limit value, the refractive index is not excessively decreased. Therefore, this configuration has an advantage in achieving a wide-angle light distribution. In addition, since a material having sufficient hardness can be selected, breakage is less likely to occur. By not allowing the corresponding value of Conditional Expression (3) to be equal to or greater than the upper limit value, the refractive index is not excessively increased. Therefore, this configuration is advantageous for improving the transmission efficiency.

2.3<Nd+0.0115×vd<2.55 (3)

In order to obtain more favorable characteristics, the lower limit value of Conditional Expression (3) is more preferably 2.31, further preferably 2.32, and further preferably 2.33. In order to obtain more favorable characteristics, the upper limit value of Conditional Expression (3) is more preferably 2.5, further preferably 2.45, and further preferably 2.4. For example, it is more preferable that the illumination lens L1 satisfies at least one of Conditional Expression (3-1), (3-2), or (3-3).

2.31<Nd+0.0115×vd<2.5 (3-1)

2.32<Nd+0.0115×vd<2.45 (3-2)

2.33<Nd+0.0115×vd<2.4 (3-3)

It is preferable that the illumination lens L1 satisfies Conditional Expression (4). Ensuring that a corresponding value of Conditional Expression (4) is not less than or equal to its lower limit value prevents an excessively low refractive index and thus, achieves an advantage in achieving a wide-angle of light distribution. In addition, since a material having sufficient hardness can be selected, breakage is less likely to occur. Ensuring that the corresponding value of Conditional Expression (4) is not greater than or equal to its upper limit value prevents an excessively high refractive index and thus, achieves an advantage in improving the transmission efficiency.

3.2<Nd−0.001×vd²+0.075×vd<3.6 (4)

In order to obtain more favorable characteristics, the lower limit value of Conditional Expression (4) is more preferably 3.22, further preferably 3.24, and further preferably 3.26. In order to obtain more favorable characteristics, the upper limit value of Conditional Expression (4) is more preferably 3.55, further preferably 3.5, and further preferably 3.45. For example, it is more preferable that the illumination lens L1 satisfies at least one of Conditional Expression (4-1), (4-2), or (4-3).

3.22<Nd−0.001×vd²+0.075×vd<3.55 (4-1)

3.24<Nd−0.001×vd²+0.075×vd<3.5 (4-2)

3.26<Nd−0.001×vd²+0.075×vd<3.45 (4-3)

It is preferable that the illumination lens L1 satisfies Conditional Expression (5). Here, an outermost diameter of the illumination lens L1 is represented by Y. For example, FIG. 4 shows Y/2 that is half of the diameter Y (that is, a radius of the illumination lens L1). By not allowing a corresponding value of Conditional Expression (5) to be equal to or less than the lower limit value, the outer diameter of the illumination lens L1 is not excessively decreased. Thus, an advantage in achieving a wide-angle light distribution and improving the transmission efficiency is achieved. By not allowing the corresponding value of Conditional Expression (5) to be equal to or greater than the upper limit value, the thickness of the illumination lens L1 is not excessively decreased. Thus, breakage is less likely to occur. In addition, since the refractive index is not excessively increased, an advantage of improving the transmission efficiency is achieved.

5<Nd×Y/D2<20 (5)

In order to obtain more favorable characteristics, the lower limit value of Conditional Expression (5) is more preferably 5.2, further preferably 5.5, and further preferably 5.8. In order to obtain more favorable characteristics, the upper limit value of Conditional Expression (5) is more preferably 15, further preferably 10, and further preferably 8.5. For example, it is more preferable that the illumination lens L1 satisfies at least one of Conditional Expression (5-1), (5-2), or (5-3).

5.2<Nd×Y/D2<15 (5-1)

5.5<Nd×Y/D2<10 (5-2)

5.8<Nd×Y/D2<8.5 (5-3)

It is preferable that the illumination lens L1 satisfies Conditional Expression (6). Here, it is assumed that a paraxial curvature radius of the second lens surface 20 is R2. By setting the corresponding value of Conditional Expression (6) to 1 or more of the lower limit value, it is easy to increase the negative optical power of the first lens surface 10, and this configuration is advantageous in achieving wide light distribution. In addition, in a case where the corresponding value of Conditional Expression (6) is set to 1 or more, the second lens surface 20 is a convex surface or a plane. Therefore, in a case where the illumination lens L1 is used as an endoscope, it is difficult for an adherent such as a residue or a water droplet to remain on the lens surface, and it is possible to suppress blocking of a ray or a change in light distribution due to the adherent. By not allowing the corresponding value of Conditional Expression (6) to be equal to or greater than the upper limit value, excessive spread of the ray is suppressed, blocking of the ray on the outer peripheral surface is suppressed, and this configuration is advantageous in improving the transmission efficiency.

1≤(R1+R2)/(R2−R1)<1.26 (6)

In order to obtain more good characteristics, the lower limit value of Conditional Expression (6) is more preferably 1.03, still more preferably 1.05, and even more preferably 1.07. In order to obtain more favorable characteristics, the upper limit value of Conditional Expression (6) is more preferably 1.23, further preferably 1.2, and further preferably 1.17. For example, it is more preferable that the illumination lens L1 satisfies at least one of Conditional Expression (6-1), (6-2), or (6-3).

1.03<(R2+R1)/(R2−R1)<1.23 (6-1)

1.05<(R2+R1)/(R2−R1)<1.2 (6-2)

1.07<(R2+R1)/(R2−R1)<1.17 (6-3)

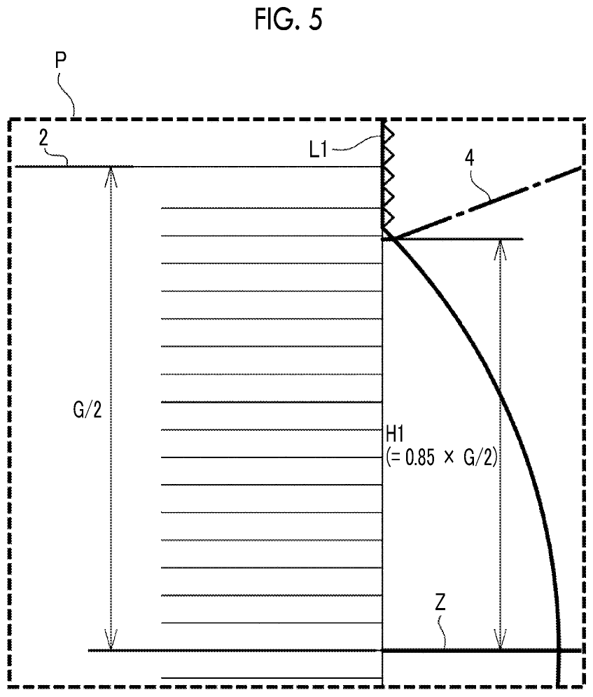

It is preferable that the illumination lens L1 satisfies Conditional Expression (7). Here, a height H1 from the optical axis Z is defined as H1=0.85×G/2. An angle with respect to the optical axis Z at which a ray 4 emitted parallel to the optical axis Z from the emission end surface of the light guide 2 at the height H1 exits the second lens surface 20 is denoted by φ2. The unit of φ2 is radian. For example, FIG. 4 shows the height H1, the angle φ2, and the ray 4. FIG. 5 shows a partially enlarged view of a region P in FIG. 4, and shows the height H1, a radius G/2 of the light guide 2, and the ray 4 as an example. By not allowing a corresponding value of Conditional Expression (7) to be equal to or less than the lower limit value, the curvature radius of the first lens surface 10 is not excessively increased. Therefore, it is easy to increase the negative optical power, and there is an advantage in achieving wide light distribution. By not allowing the corresponding value of Conditional Expression (7) to be equal to or greater than the upper limit value, excessive spread of the ray is suppressed, and the ray is suppressed from being blocked on the outer peripheral surface. There is an advantage in improving the transmission efficiency.

0.25<φ2×H1/|R1|<0.8 (7)

In order to obtain more favorable characteristics, the lower limit value of Conditional Expression (7) is more preferably 0.3, further preferably 0.35, and further preferably 0.4. In order to obtain more favorable characteristics, the upper limit value of Conditional Expression (7) is more preferably 0.75, further preferably 0.7, and further preferably 0.65. For example, it is more preferable that the illumination lens L1 satisfies at least one of Conditional Expression (7-1), (7-2), or (7-3).

0.3<φ2×H1/|R1|<0.75 (7-1)

0.35<φ2×H1/|R1|<0.7 (7-2)

0.4<φ2×H1/|R1|<0.65 (7-3)

It is preferable that the illumination lens L1 satisfies Conditional Expression (8). Here, a height of the ray 4 from the optical axis Z at the second lens surface 20, the ray 4 being emitted parallel to the optical axis Z from the emission end surface of the light guide 2 at the height H1, is denoted by H2. For example, FIG. 4 shows the height H2. By not allowing the corresponding value of Conditional Expression (8) to be equal to or less than the lower limit value, the ray angle can be provided in the illumination lens L1. Thus, it is easy to increase the negative optical power, and it is advantageous to achieve wide light distribution. By not allowing the corresponding value of Conditional Expression (8) to be equal to or greater than the upper limit value, excessive spread of the ray is suppressed, and the ray is suppressed from being blocked on the outer peripheral surface. Thus, it is advantageous to improve the transmission efficiency.

1.15<H2/H1<1.8 (8)

In order to obtain more favorable characteristics, the lower limit value of Conditional Expression (8) is more preferably 1.2, further preferably 1.25, and further preferably 1.3. In order to obtain more good characteristics, the upper limit value of Conditional Expression (8) is more preferably 1.7, still more preferably 1.6, and even more preferably 1.5. For example, it is more preferable that the illumination lens L1 satisfies at least one of Conditional Expression (8-1), (8-2), or (8-3).

1.2<H2/H1<1.7 (8-1)

1.25<H2/H1<1.6 (8-2)

1.3<H2/H1<1.5 (8-3)

It is preferable that the illumination lens L1 satisfies Conditional Expression (9). By not allowing the corresponding value of Conditional Expression (9) to be equal to or less than the lower limit value thereof, the height H2 is not excessively separated from the outermost diameter of the illumination lens L1, and the outermost diameter of the illumination lens L1 is not excessively large. Thus, there is an advantage in achieving reduction in size. By not allowing the corresponding value of Conditional Expression (9) to be equal to or greater than the upper limit value thereof, the height H2 is not excessively close to the outermost diameter of the illumination lens L1. Thus, there is an advantage in suppressing blocking of rays on the outer peripheral surface and improving the transmission efficiency.

0.5<2×H2/Y<0.99 (9)

In order to obtain more favorable characteristics, the lower limit value of Conditional Expression (9) is more preferably 0.55, further preferably 0.6, and further preferably 0.65. In order to obtain more favorable characteristics, the upper limit value of Conditional Expression (9) is more preferably 0.96, further preferably 0.93, and further preferably 0.9. For example, it is more preferable that the illumination lens L1 satisfies at least one of Conditional Expression (9-1), (9-2), or (9-3).

0.55<2×H2/Y<0.96 (9-1)

0.6<2×H2/Y<0.93 (9-2)

0.65<2×H2/Y<0.9 (9-3)

It is preferable that the illumination lens L1 satisfies Conditional Expression (10). Here, it is assumed that a center thickness of the illumination lens L1 is T. It is assumed that a distance in a direction of the optical axis Z from an intersection of a ray 4 emitted parallel to the optical axis Z from an emission end surface of the light guide 2 at a height H1 to an intersection with the second lens surface 20 with respect to a first lens surface 10 is E. For example, FIG. 4 shows the center thickness T and the distance E. By not allowing a corresponding value of Conditional Expression (10) to be equal to or less than the lower limit value, the center thickness T of the illumination lens L1 is not excessively increased. Thus, blocking of the ray on the outer peripheral surface is suppressed, and an advantage of improving the transmission efficiency is achieved. By not allowing the corresponding value of Conditional Expression (10) to be equal to or greater than the upper limit value, the center thickness T of the illumination lens L1 is not excessively decreased. Thus, breakage is less likely to occur.

1.3<E/T<1.9 (10)

In order to obtain more favorable characteristics, the lower limit value of Conditional Expression (10) is more preferably 1.35, further preferably 1.4, and further preferably 1.45. In order to obtain more favorable characteristics, the upper limit value of Conditional Expression (10) is more preferably 1.85, further preferably 1.8, and further preferably 1.75. For example, it is more preferable that the illumination lens L1 satisfies at least one of Conditional Expression (10-1), (10-2), or (10-3).

1.35<E/T<1.85 (10-1)

1.4<E/T<1.8 (10-2)

1.45<E/T<1.75 (10-3)

It is preferable that the illumination lens L1 satisfies Conditional Expressions (11) to (13). Here, the wear degree of the illumination lens L1 is denoted by Aa. By setting the corresponding values of Conditional Expressions (11) to (13) within the ranges of Conditional Expressions (11) to (13), the illumination lens L1 can be formed of a material having a high refractive index and a small wear degree (that is, a hard material). Therefore, it is possible to reduce the thickness while preventing breakage, which is advantageous for achieving reduction in size, wide light distribution, and improvement in transmission efficiency.

1.7<Nd<2.1 (11)

25<vd<50 (12)

50<Aa<90 (13)

The wear degree is an indicator representing mechanical properties of a material and is calculated based on a volume decrease amount in a case where the material is worn by using an abrasive. A value of the wear degree of glass is generally about 50 to 500, and a smaller value means that the glass is harder. The wear degree also has a high correlation with other hardness indices such as a Knoop hardness.

The preferable configurations and available configurations described above, also including configurations related to conditional expressions, may be optionally combined without contradiction, and it is preferable to selectively adopt configurations in accordance with required specifications as appropriate.

For example, a preferred aspect of the illumination lens according to the present disclosure is an illumination lens that is disposed at a distal end of a light guide of an endoscope, in which the illumination lens consists of one negative lens, a first lens surface of the illumination lens on a light guide side includes a concave surface in a center portion and a light diffusion surface on a radial outside of the concave surface, and Conditional Expression (1) is satisfied.

Next, examples of the illumination lens according to the present disclosure will be described with reference to the drawings. Reference numerals provided in the cross-sectional view of each example are independently used for each example in order to avoid complication of description and the drawings caused by an increase in the number of digits of the reference numerals. Accordingly, a common reference numeral provided in the drawings of different examples does not necessarily indicate a common configuration.

### Example 1

A cross-sectional view of a configuration of an illumination lens L1 of Example 1 is shown in FIG. 1, and a method of showing the cross-sectional view is as described above. Therefore, duplicate description will be partially omitted. The illumination lens L1 of Example 1 consists of one negative lens. A first lens surface 10 on a light guide 2 side includes a concave surface 10a in a center portion and a light diffusion surface 10b on a radial outside of the concave surface 10a. The above is an outline of the illumination lens L1 of Example 1.

For the illumination lens L1 of Example 1, basic lens data is shown in Table 1, and specifications are shown in Table 2. The table of the basic lens data is described as below. A column of Sn shows a surface number in a case where an emission end surface of the light guide 2 is set as a first surface and the number is increased by one toward the irradiated object side. A curvature radius of each surface is shown in a column of R. A surface spacing on the optical axis between each surface and a surface, which is adjacent to an irradiated object side of the surface, is shown in a column of D. A column of Nd shows a refractive index of the lens at a d line, and a column of vd shows an Abbe number of the lens based on the d line. A column of Aa shows a wear degree of the lens.

Table 2 shows a diameter H of the concave surface 10a on the first lens surface 10, a diameter G of the emission end surface of the light guide 2, a diameter Y of the outermost diameter of the illumination lens L1, and a focal length f of the illumination lens L1 based on the d line.

Millimeters are used as a unit of length in the data of each table. However, since the optical system can be used even though the optical system is proportionally enlarged or reduced, other appropriate units can be used. In addition, the numerical values rounded to predetermined digits are described in each table shown below.

TABLE 1
Example 1

| Sn | R | D | Nd | vd | Aa |
|---|---|---|---|---|---|
| 1 | | 0.1200 | | | |
| 2 | −0.4500 | 0.2000 | 2.001 | 29.13 | 55 |
| 3 | −6.5073 | | | | |

TABLE 2
Example 1

| | |
|---|---|
| H | 0.612 |
| G | 0.70 |
| Y | 1.15 |
| f | −0.491 |

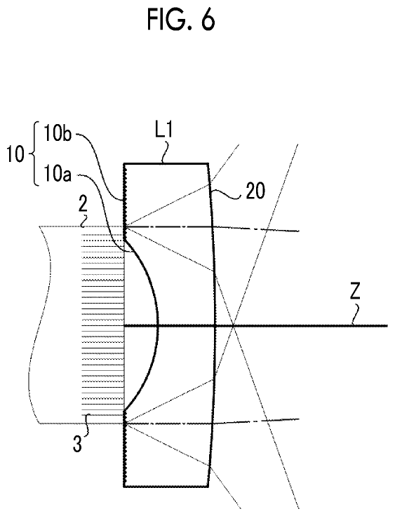

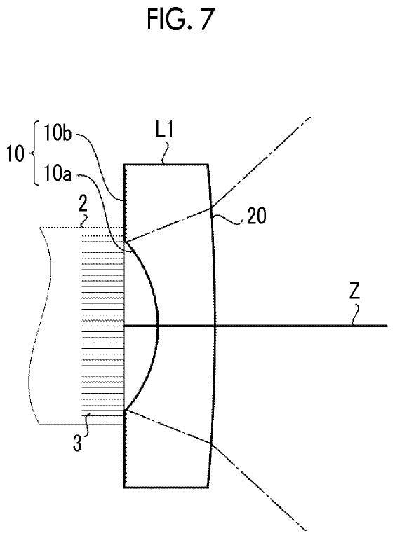

FIGS. 6 and 7 are cross-sectional views showing a configuration and an optical path of the illumination lens L1 of Example 1. The optical path shown in FIG. 6 is an optical path of rays in a case where a plurality of rays emitted from a plurality of points on the emission end surface of the light guide 2 are incident on the light diffusion surface 10b of the illumination lens L1. The optical path shown in FIG. 7 is an optical path of rays emitted parallel to the optical axis Z from the emission end surface of the light guide 2 at a height H1. Since methods of illustrating the cross-sectional views in FIGS. 6 and 7 are the same as that in FIG. 1, repeated description will be omitted here.

Symbols, meanings, description methods, and illustration methods of the respective data pieces according to Example 1 are the same as those in the following examples unless otherwise specified. Therefore, in the following description, repeated description will not be given.

### Example 2

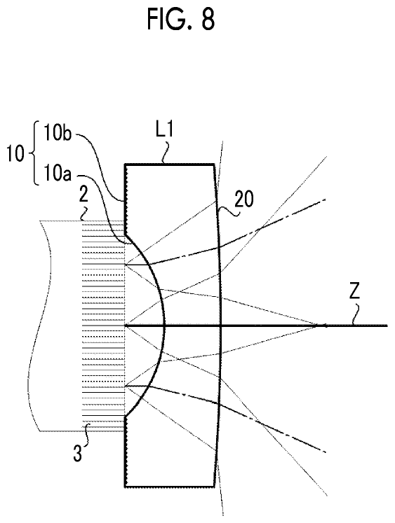

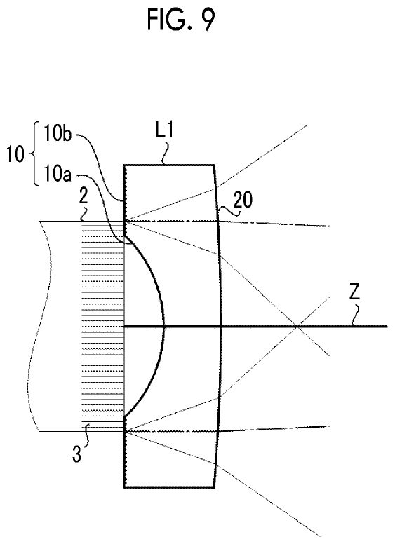

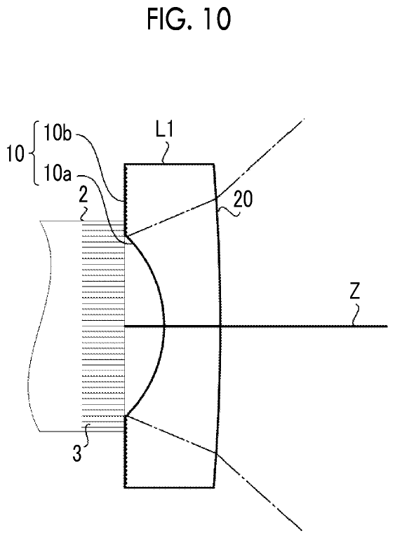

FIGS. 8 to 10 are cross-sectional views showing a configuration and an optical path of the illumination lens L1 of Example 2. FIG. 8 shows an optical path of rays incident on the concave surface 10a. FIG. 9 shows an optical path of rays incident on the light diffusion surface 10b. FIG. 10 shows an optical path of rays emitted at a height H1. The illumination lens L1 of Example 2 has the same configuration as the outline of the illumination lens L1 of Example 1. Regarding the illumination lens L1 of Example 2, Table 3 shows basic lens data, and Table 4 shows specifications thereof.

TABLE 3
Example 2

| Sn | R | D | Nd | vd | Aa |
|---|---|---|---|---|---|
| 1 | | 0.1400 | | | |
| 2 | −0.4500 | 0.2000 | 1.883 | 40.78 | 60 |
| 3 | −7.0044 | | | | |

TABLE 4
Example 2

| | |
|---|---|
| H | 0.652 |
| G | 0.75 |
| Y | 1.15 |
| f | −0.553 |

### Example 3

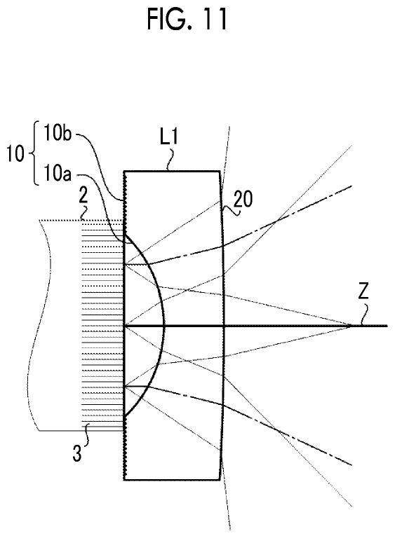

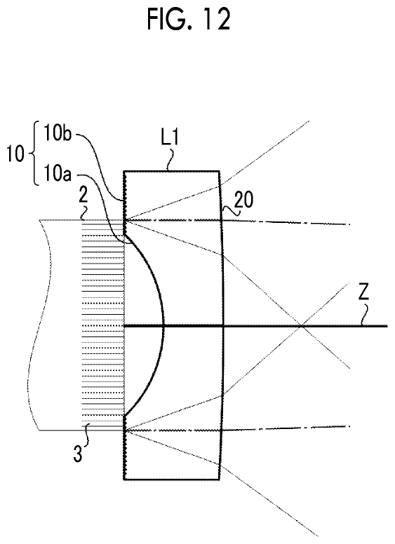

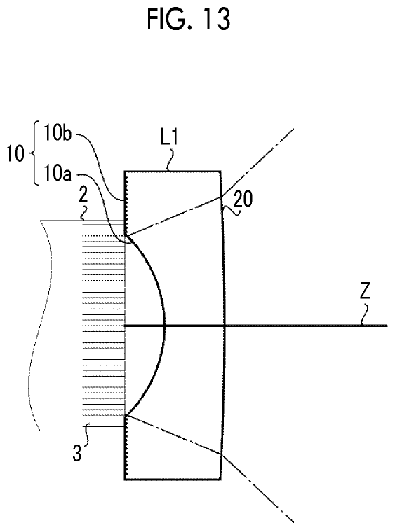

FIGS. 11 to 13 are cross-sectional views showing a configuration and an optical path of the illumination lens L1 of Example 3. FIG. 11 shows an optical path of rays incident on the concave surface 10a. FIG. 12 shows an optical path of rays incident on the light diffusion surface 10b. FIG. 13 shows an optical path of rays emitted at a height H1. The illumination lens L1 of Example 3 has the same configuration as the outline of the illumination lens L1 of Example 1. Regarding the illumination lens L1 of Example 3, Table 5 shows basic lens data, and Table 6 shows specifications thereof.

TABLE 5
Example 3

| Sn | R | D | Nd | vd | Aa |
|---|---|---|---|---|---|
| 1 | | 0.1400 | | | |
| 2 | −0.4500 | 0.2100 | 1.883 | 40.78 | 60 |
| 3 | −9.9601 | | | | |

TABLE 6
Example 3

| | |
|---|---|
| H | 0.652 |
| G | 0.75 |
| Y | 1.10 |
| f | −0.539 |

### Example 4

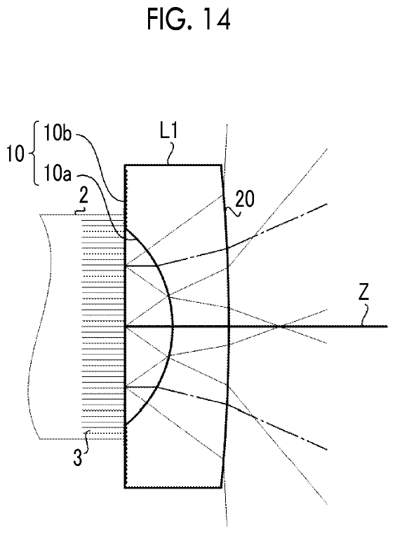

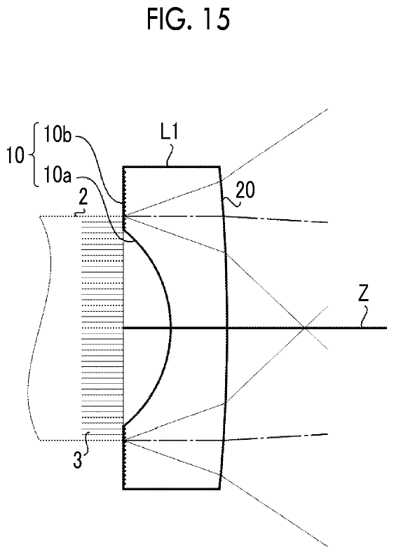

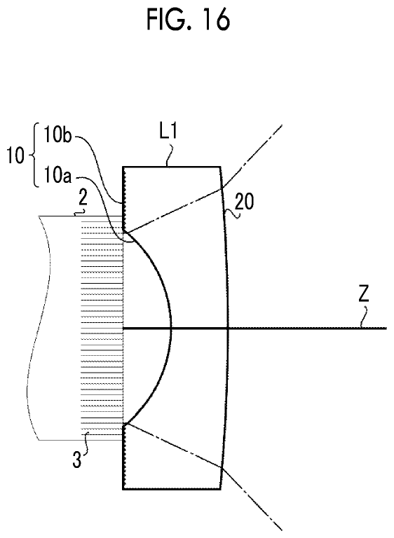

FIGS. 14 to 16 are cross-sectional views showing a configuration and an optical path of the illumination lens L1 of Example 4. FIG. 14 shows an optical path of rays incident on the concave surface 10a. FIG. 15 shows an optical path of rays incident on the light diffusion surface 10b. FIG. 16 shows an optical path of rays emitted at a height H1. The illumination lens L1 of Example 4 has the same configuration as the outline of the illumination lens L1 of Example 1. Regarding the illumination lens L1 of Example 4, Table 7 shows basic lens data, and Table 8 shows specifications thereof.

TABLE 7
Example 4

| Sn | R | D | Nd | vd | Aa |
|---|---|---|---|---|---|
| 1 | | 0.1700 | | | |
| 2 | −0.4500 | 0.2000 | 1.883 | 40.78 | 60 |
| 3 | −6.0189 | | | | |

TABLE 8
Example 4

| | |
|---|---|
| H | 0.705 |
| G | 0.80 |
| Y | 1.15 |
| f | −0.560 |

### Example 5

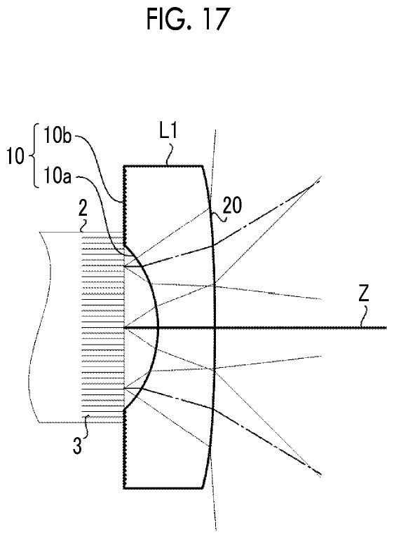

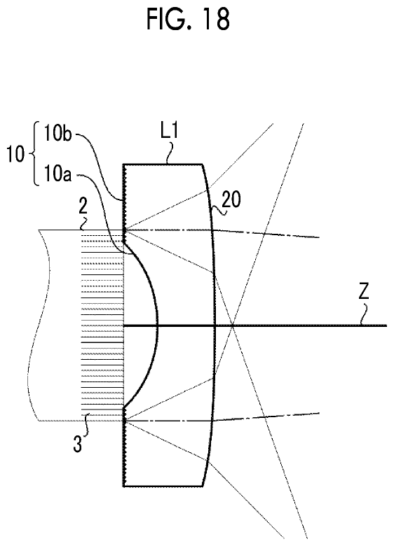

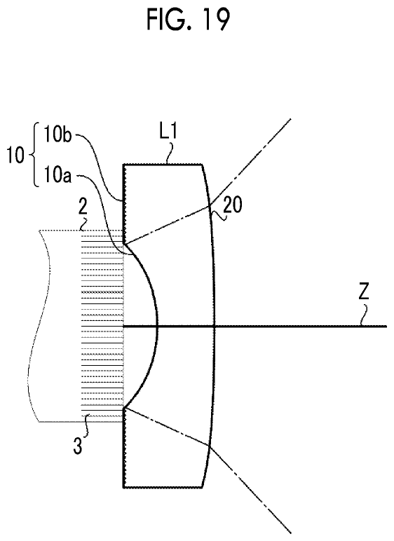

FIGS. 17 to 19 are cross-sectional views showing a configuration and an optical path of the illumination lens L1 of Example 5. FIG. 17 shows an optical path of rays incident on the concave surface 10a. FIG. 18 shows an optical path of rays incident on the light diffusion surface 10b. FIG. 19 shows an optical path of rays emitted at a height H1. The illumination lens L1 of Example 5 has the same configuration as the outline of the illumination lens L1 of Example 1. Regarding the illumination lens L1 of Example 5, Table 9 shows basic lens data, and Table 10 shows specifications thereof.

In the basic lens data, a reference sign * is attached to a surface number of the aspherical surface, and a value of a paraxial curvature radius is written in a field of a curvature radius of the aspherical surface. In Table 11, the surface number of the aspherical surface is written in a row of Sn and numerical values of aspherical coefficients of respective aspherical surfaces are written in rows of KA and Am (m=4, 6). The “E±n” (n: integer) in numerical values of the aspherical coefficients in Table 11 indicates “×10^{±n}”. KA and Am are the aspherical coefficients in an aspheric equation represented by the following expression.

Zd=C×h²/{1+(1−KA×C²×h²)^{1/2}}+ΣAm×h^{m}

where,
Zd: a depth of the aspherical surface (a length of a perpendicular line drawn from a point on the aspherical surface at a height h to a plane that is in contact with an aspherical surface apex and that is perpendicular to the optical axis Z),
h: a height (a distance from the optical axis Z to the lens surface),
C: a reciprocal of the paraxial curvature radius,
KA, Am: aspherical coefficients,
Σ in the aspheric equation means a sum related to m.

TABLE 9
Example 5

| Sn | R | D | Nd | vd | Aa |
|---|---|---|---|---|---|
| 1 | | 0.1200 | | | |
| 2* | −0.4500 | 0.2000 | 2.051 | 26.94 | 50 |
| 3* | −6.5073 | | | | |

TABLE 10
Example 5

| | |
|---|---|
| H | 0.589 |
| G | 0.68 |
| Y | 1.15 |
| f | −0.468 |

TABLE 11
Example 5

| Sn | 2 | 3 |
|---|---|---|
| KA | 1.0000000E + 00 | 1.0000000E + 00 |
| A4 | −5.0000000E − 01 | −1.0000000E − 02 |
| A6 | −1.0000000E + 01 | −5.0000000E − 01 |

### Example 6

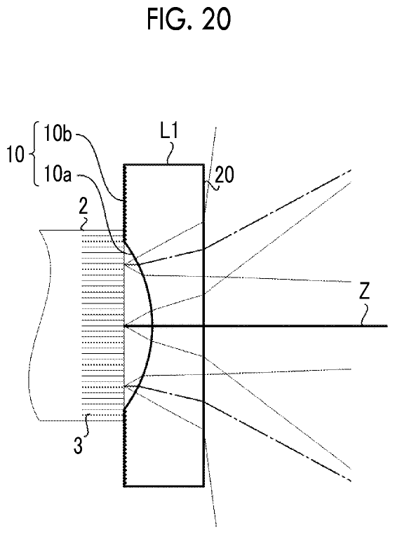

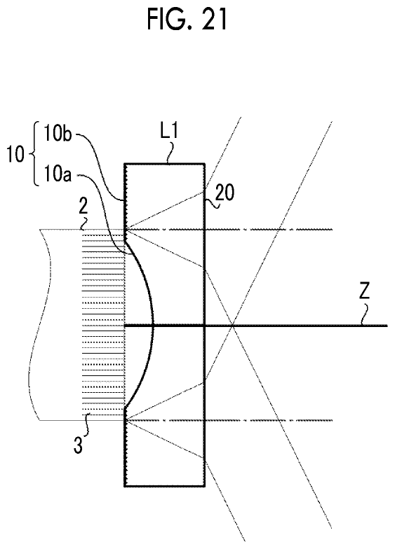

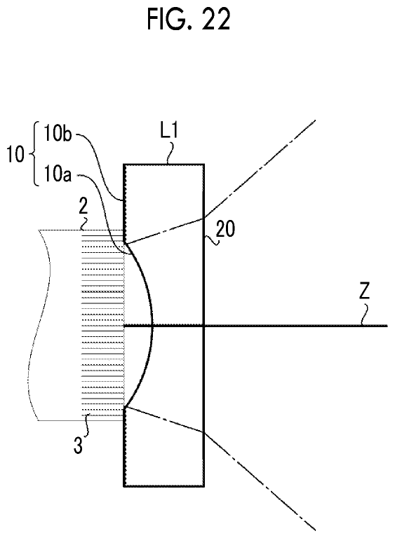

FIGS. 20 to 22 are cross-sectional views showing a configuration and optical paths of the illumination lens L1 of Example 6. FIG. 20 shows optical paths of rays incident on the concave surface 10a. FIG. 21 shows optical paths of rays incident on the light diffusion surface 10b. FIG. 22 shows optical paths of rays emitted at the height H1. The illumination lens L1 of Example 6 has the same configuration as the outline of the illumination lens L1 of Example 1. Regarding the illumination lens L1 of Example 6, Table 12 shows basic lens data, and Table 13 shows specifications.

TABLE 12
Example 6

| Sn | R | D | Nd | vd | Aa |
|---|---|---|---|---|---|
| 1 | | 0.1000 | | | |
| 2 | −0.5000 | 0.1800 | 2.051 | 26.94 | 50 |
| 3 | 00 | | | | |

TABLE 13
Example 6

| | |
|---|---|
| H | 0.600 |
| G | 0.68 |
| Y | 1.15 |
| f | −0.476 |

Table 14 shows corresponding values of Conditional Expressions (1) to (10) of the illumination lenses L1 of Examples 1 to 6. Preferable ranges of the conditional expressions may be set using the corresponding values of the examples shown in table 14 as upper limit values or lower limit values of the conditional expressions.

TABLE 14

| Expression | | | | |
|---|---|---|---|---|
| Number | Conditional Expression | Example 1 | Example 2 | Example 3 |
| (1) | Nd × G/D2 | 4.38 | 4.15 | 4.03 |
| (2) | $\frac{2 \sqrt{{\vert R 1 \vert}^{2} - {( \vert R 1 \vert - D 1 )}^{2}}}{G}$ | 0.87 | 0.87 | 0.87 |
| (3) | Nd + 0.0115 × vd | 2.34 | 2.35 | 2.35 |
| (4) | Nd − 0.001 × vd² + 0.075 × vd | 3.34 | 3.28 | 3.28 |
| (5) | Nd × Y/D2 | 7.19 | 6.37 | 5.92 |
| (6) | (R2 + R1)/(R2 − R1) | 1.15 | 1.14 | 1.09 |
| (7) | φ2 × H1/\|R1\| | 0.49 | 0.52 | 0.54 |
| (8) | H2/H1 | 1.41 | 1.42 | 1.44 |
| (9) | 2 × H2/Y | 0.73 | 0.79 | 0.84 |
| (10) | E/T | 1.49 | 1.59 | 1.58 |
| Expression | | | | |
| Number | Conditional Expression | Example 4 | Example 5 | Example 6 |
| (1) | NdxG/D2 | 4.07 | 4.36 | 4.98 |
| (2) | $\frac{2 \sqrt{{\vert R 1 \vert}^{2} - {( \vert R 1 \vert - D 1 )}^{2}}}{G}$ | 0.88 | 0.90 | 0.88 |
| (3) | Nd + 0.0115 × vd | 2.35 | 2.36 | 2.36 |
| (4) | Nd − 0.001 × vd² + 0.075 × vd | 3.28 | 3.35 | 3.35 |
| (5) | Nd × Y/D2 | 5.85 | 7.37 | 8.42 |
| (6) | (R2 + R1)/(R2 − R1) | 1.16 | 1.15 | 1.00 |
| (7) | φ2 × H1/\|R1\| | 0.61 | 0.48 | 0.42 |
| (8) | H2/H1 | 1.47 | 1.40 | 1.32 |
| (9) | 2 × H2/Y | 0.87 | 0.71 | 0.66 |
| (10) | E/T | 1.67 | 1.46 | 1.51 |

As described above, all of the illumination lenses L1 of Examples 1 to 6 are configured to be small, and have a wide light distribution angle and a good transmission efficiency.

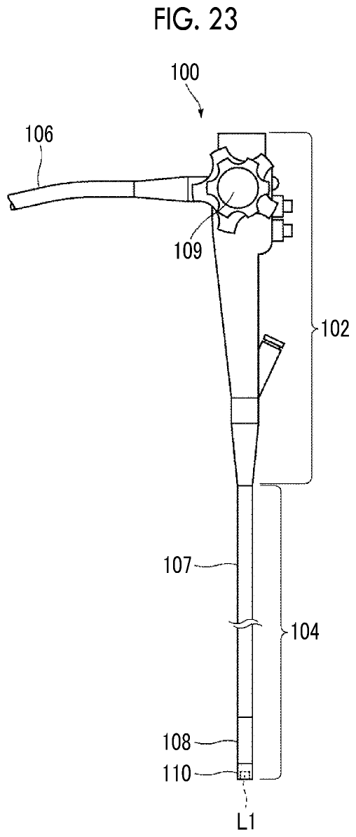

Next, an endoscope according to the embodiment of the present disclosure will be described. FIG. 23 shows a diagram showing a schematic configuration of the entire endoscope according to the embodiment of the disclosure. The endoscope 100 shown in FIG. 23 mainly comprises an operation part 102, an insertion part 104, and a universal cord 106 that is to be connected to a connector part (not shown). A large portion of the insertion part 104 is a flexible part 107 that is bendable in any direction along an insertion path, a bendable part 108 is connected to a distal end of the flexible part 107, and a distal end part 110 is connected to a distal end of the bendable part 108. The bendable part 108 is provided to allow the distal end part 110 to face a desired direction and can be operated to be bent by the rotational movement of a bending operation knob 109 provided at the operating part 102.

The illumination lens L1 according to the embodiment of the present disclosure is disposed at an internal distal end of the distal end part 110. FIG. 23 schematically shows the illumination lens L1. Since the endoscope according to the embodiment of the present disclosure comprises the illumination lens L1 according to the embodiment of the present disclosure, observation can be performed using illumination light with a wide angle while achieving miniaturization of the distal end part of the insertion part 104.

The technique of the present disclosure has been described above using the embodiment and Examples. However, the technique of the present disclosure is not limited to the embodiment and Examples described above, and may be modified in various ways. For example, the thickness, the curvature radius, the surface spacing, the refractive index, the Abbe number, the aspherical coefficient, and the like of each constituent element are not limited to the values shown in each of the above-described examples, and other values can be used.

Regarding the above-described embodiments and examples, the following Supplementary Notes will be further disclosed.

### Supplementary Note 1

An illumination lens that is disposed at a distal end of a light guide of an endoscope, the illumination lens consisting of
one negative lens,
in which a first lens surface of the illumination lens on a light guide side includes a concave surface that is provided at a center portion thereof and a light diffusion surface that is provided outside the concave surface in a radial direction, and
in a case where
   a refractive index of the illumination lens at a d line is denoted by Nd,
   a diameter of the light guide is denoted by G, and
   a distance on an optical axis from an emission end surface of the light guide to a second lens surface of the illumination lens on an irradiated object side is denoted by D2,
Conditional Expression (1) is satisfied, which is represented by 3.55<Nd×G/D2<10 (1).

### Supplementary Note 2

The illumination lens according to Supplementary Note 1,
in which in a case where
   a paraxial curvature radius of the first lens surface is denoted by R1, and
   a distance on the optical axis from the emission end surface of the light guide to the first lens surface is denoted by D1,
Conditional Expression (2) is satisfied, which is represented by

$$0.82 < \frac{2 \sqrt{{\vert R 1 \vert}^{2} - {( \vert R 1 \vert - D 1 )}^{2}}}{G} < 0.95 . \qquad ( 2 )$$

### Supplementary Note 3

The illumination lens according to Supplementary Note 1 or 2,
in which in a case where an Abbe number of the illumination lens based on the d line is denoted by vd,
Conditional Expression (3) is satisfied, which is represented by 2.3<Nd+0.0115×vd<2.55 (3).

### Supplementary Note 4

The illumination lens according to any one of Supplementary Notes 1 to 3,
in which in a case where an Abbe number of the illumination lens based on the d line is denoted by vd,
Conditional Expression (4) is satisfied, which is represented by 3.2<Nd−0.001×vd²+0.075×vd<3.6 (4).

### Supplementary Note 5

The illumination lens according to any one of Supplementary Notes 1 to 4,
in which in a case where an outermost diameter of the illumination lens is denoted by Y,
Conditional Expression (5) is satisfied, which is represented by 5<Nd×Y/D2<20 (5).

### Supplementary Note 6

The illumination lens according to any one of Supplementary Notes 1 to 5,
in which in a case where
   a paraxial curvature radius of the first lens surface is denoted by R1, and
   a paraxial curvature radius of the second lens surface is denoted by R2,
Conditional Expression (6) is satisfied, which is represented by 1≤(R2+R1)/(R2−R1)<1.26 (6).

### Supplementary Note 7

The illumination lens according to any one of Supplementary Notes 1 to 6,
in which in a case where
   a height from the optical axis, denoted by H1, is defined as H1=0.85×G/2,
   an angle with respect to the optical axis at which a ray emitted parallel to the optical axis from the emission end surface of the light guide at the height H1 exits the second lens surface is denoted by φ2,
   a paraxial curvature radius of the first lens surface is denoted by R1, and
   a unit of φ2 is radian,
Conditional Expression (7) is satisfied, which is represented by 0.25<Y2×H1/|R1|<0.8 (7).

### Supplementary Note 8

The illumination lens according to any one of Supplementary Notes 1 to 7,
in which in a case where
   a height from the optical axis, denoted by H1, is defined as H1=0.85×G/2, and
   a height of a ray from the optical axis at the second lens surface, the ray being emitted parallel to the optical axis from the emission end surface of the light guide at the height H1, is denoted by H2,
Conditional Expression (8) is satisfied, which is represented by 1.15<H2/H1<1.8 (8).

### Supplementary Note 9

The illumination lens according to any one of Supplementary Notes 1 to 8,
in which in a case where
   a height from the optical axis, denoted by H1, is defined as H1=0.85×G/2,
   a height of a ray from the optical axis at the second lens surface, the ray being emitted parallel to the optical axis from the emission end surface of the light guide at the height H1, is denoted by H2, and
an outermost diameter of the illumination lens is denoted by Y,
Conditional Expression (9) is satisfied, which is represented by 0.5<2×H2/Y<0.99 (9).

### Supplementary Note 10

The illumination lens according to any one of Supplementary Notes 1 to 9,
in which in a case where
a center thickness of the illumination lens is denoted by T,
a height from the optical axis, denoted by H1, is defined as H1=0.85×G/2, and
a distance in a direction of the optical axis from an intersection of a ray emitted parallel to the optical axis from the emission end surface of the light guide at the height H1 with the first lens surface to an intersection of the ray with the second lens surface is denoted by E,
Conditional Expression (10) is satisfied, which is represented by 1.3<E/T<1.9 (10).

### Supplementary Note 11

An endoscope including:
the illumination lens according to any one of Supplementary Notes 1 to 10.

What is claimed is:

1. An illumination lens that is disposed at a distal end of a light guide of an endoscope, the illumination lens consisting of one negative lens, wherein:
a first lens surface of the illumination lens on a light guide side includes a concave surface that is provided at a center portion thereof and a light diffusion surface that is provided outside the concave surface in a radial direction, and
in a case where
a refractive index of the illumination lens at a d line is denoted by Nd,
a diameter of the light guide is denoted by G, and
a distance on an optical axis from an emission end surface of the light guide to a second lens surface of the illumination lens on an irradiated object side is denoted by D2,
Conditional Expression (1) is satisfied:
3.55<Nd×G/D2<10 (1).

2. The illumination lens according to claim 1, wherein
in a case where
a paraxial curvature radius of the first lens surface is denoted by R1, and
a distance on the optical axis from the emission end surface of the light guide to the first lens surface is denoted by D1,
Conditional Expression (2) is satisfied:
$$0.82 < \frac{2 \sqrt{{\vert R 1 \vert}^{2} - {( \vert R 1 \vert - D 1 )}^{2}}}{G} < 0 \text{.95} \qquad ( 2 )$$

3. The illumination lens according to claim 1, wherein
in a case where an Abbe number of the illumination lens based on the d line is denoted by vd,
Conditional Expression (3) is satisfied:
2.3<Nd+0.0115×vd<2.55 (3).

4. The illumination lens according to claim 1, wherein
in a case where an Abbe number of the illumination lens based on the d line is denoted by vd,
Conditional Expression (4) is satisfied:
3.2<Nd−0.001×vd²+0.075×vd<3.6 (4).

5. The illumination lens according to claim 1, wherein
in a case where an outermost diameter of the illumination lens is denoted by Y,
Conditional Expression (5) is satisfied:
5<Nd×Y/D2<20 (5).

6. The illumination lens according to claim 1, wherein
in a case where
a paraxial curvature radius of the first lens surface is denoted by R1, and
a paraxial curvature radius of the second lens surface is denoted by R2,
Conditional Expression (6) is satisfied:
1≤(R2+R1)/(R2−R1)≤1.26 (6).

7. The illumination lens according to claim 1, wherein
in a case where
a height from the optical axis, denoted by H1, is defined as H1=0.85×G/2,
an angle with respect to the optical axis at which a ray emitted parallel to the optical axis from the emission end surface of the light guide at the height H1 exits the second lens surface is denoted by φ2,
a paraxial curvature radius of the first lens surface is denoted by R1, and
a unit of φ2 is radian,
Conditional Expression (7) is satisfied:
0.25<φ2×H1/|R1|<0.8 (7).

8. The illumination lens according to claim 1, wherein
in a case where
a height from the optical axis, denoted by H1, is defined as H1=0.85×G/2, and
a height of a ray from the optical axis at the second lens surface, the ray being emitted parallel to the optical axis from the emission end surface of the light guide at the height H1, is denoted by H2,
Conditional Expression (8) is satisfied:
1.15<H2/H1<1.8 (8).

9. The illumination lens according to claim 1, wherein
in a case where
a height from the optical axis, denoted by H1, is defined as H1=0.85×G/2,
a height of a ray from the optical axis at the second lens surface, the ray being emitted parallel to the optical axis from the emission end surface of the light guide at the height H1, is denoted by H2, and
an outermost diameter of the illumination lens is denoted by Y,
Conditional Expression (9) is satisfied:
0.5<2×H2/Y<0.99 (9).

10. The illumination lens according to claim 1, wherein
in a case where
a center thickness of the illumination lens is denoted by T,
a height from the optical axis, denoted by H1, is defined as H1=0.85×G/2, and
a distance in a direction of the optical axis from an intersection of a ray emitted parallel to the optical axis from the emission end surface of the light guide at the height H1 with the first lens surface to an intersection of the ray with the second lens surface is denoted by E,
Conditional Expression (10) is satisfied:
1.3<E/T<1.9 (10).

11. The illumination lens according to claim 1, wherein
in a case where
a paraxial curvature radius of the first lens surface is denoted by R1,
a distance on the optical axis from the emission end surface of the light guide to the first lens surface is denoted by D1,
an Abbe number of the illumination lens based on the d line is denoted by vd,
an outermost diameter of the illumination lens is denoted by Y,
a paraxial curvature radius of the second lens surface is denoted by R2,
a height from the optical axis, denoted by H1, is defined as H1=0.85×G/2,
an angle with respect to the optical axis at which a ray emitted parallel to the optical axis from the emission end surface of the light guide at the height H1 exits the second lens surface is denoted by φ2,
a unit of φ2 is radian,
a height of a ray from the optical axis at the second lens surface, the ray being emitted parallel to the optical axis from the emission end surface of the light guide at the height H1, is denoted by H2,
a center thickness of the illumination lens is denoted by T, and
a distance in a direction of the optical axis from an intersection of a ray emitted parallel to the optical axis from the emission end surface of the light guide at the height H1 with the first lens surface to an intersection of the ray with the second lens surface is denoted by E,
Conditional Expressions (2) to (10) are satisfied:
$$0.82 < \frac{2 \sqrt{{\vert R 1 \vert}^{2} - {( \vert R 1 \vert - D 1 )}^{2}}}{G} < 0.95 \qquad ( 2 )$$
$$2.3 < Nd + 0.0 \; 115 \times ν d < 2.55 \qquad ( 3 )$$
$$3.2 < Nd - 0.0 \; 01 \times ν {d}^{2} + 0.075 \times ν d < 3.6 \qquad ( 4 )$$
$$5 < Nd \times Y / D 2 < 20 \qquad ( 5 )$$
$$1 \leq ( R 1 + R 2 ) / ( R 2 - R 1 ) < 1.26 \qquad ( 6 )$$
$$0.25 < φ 2 \times H 1 / \vert R 1 \vert < 0.8 \qquad ( 7 )$$
$$1.15 < H 2 / H 1 < 1.8 \qquad ( 8 )$$
$$0.5 < 2 \times H 2 / Y < 0.99 \qquad ( 9 )$$
$$1.3 < E / T < 1.9 \qquad ( 10 )$$

12. An endoscope comprising the illumination lens according to claim 1.
